(19) United States
(12) Patent Application Publication
(10) Pub. No.: US 2013/0244296 A1
(43) Pub. Date: Sep. 19, 2013

(54) HALOGENATION ENZYMES

(71) Applicant: Jixun Zhan, North Logan, UT (US)

(72) Inventor: Jixun Zhan, North Logan, UT (US)

(21) Appl. No.: 13/602,136

(22) Filed: Sep. 1, 2012

Related U.S. Application Data
(60) Provisional application No. 61/529,952, filed on Sep. 1, 2011.

(51) Int. Cl. C12N 9/00; C12P 17/08; C12P 7/26; C12P 17/18

(52) U.S. Cl. CPC C12N 9/0071; C12P 17/181; C12P 17/08; C12P 7/26
USPC 435/119; 435/189; 536/23.2; 435/320.1; 435/124; 435/148

(57) ABSTRACT

The present disclosure relates to an isolated or purified nucleotide sequence comprising a cDNA sequence of an rdc2 at least 60%, 70%, 90%, 95%, 98%, or 100% identical to SEQ ID NO. 1. In a second embodiment, the invention provides for a flavin-dependent halogenase comprising an amino acid sequence of an Rdc2 halogenase at least 60%, 70%, 90%, 95%, 98%, or 100% identical to SEQ ID NO. 2. In related embodiments there are provided herein methods for halogenating compounds by using an Rdc2 halogenase of the present invention.

### CROSS REFERENCE TO RELATED APPLICATIONS

This application claims priority to U.S. Provisional Application 61/529,952, filed Sep. 1, 2011, which is hereby incorporated by reference herein in its entirety.

### BACKGROUND

The present invention is in the technical field of biotechnology. More particularly, the present invention is in the technical field of enzymatic halogenation.

Halogenated molecules represent an important class of natural products, many of which are pharmaceutically relevant, such as chloramphenicol (antibacterial), vancomycin (antibacterial), and rebeccamycin (anticancer). Flavin-dependent halogenases have been identified as a major player in the introduction of halogen into activated organic molecules in natural product biosynthesis. However, flavin-dependent halogenases identified so far are mainly prokaryotic tryptophan halogenases with strict substrate specificity. Most of these enzymes are involved in early biosynthetic steps of natural products to modify precursors such as tryptophan, which has limited their potential as biocatalysts to prepare various halogenated molecules.

Biotransformation has been employed as an effective method to generate new derivatives from natural products. Compared to chemical synthesis, biotransformation has many advantages such as mild conditions, high regio- and stereoselectivity, and good efficiency. Beauveria bassiana ATCC 7159 is known to be a powerful biocatalyst that has frequently been employed in biotransformation studies due to its abundant enzyme systems, broad substrate acceptability, and its ability to perform diverse types of reactions. It has been reported that this fungus is capable of biotransformation of over 300 different substrates, involving a variety of reactions, such as hydroxylation, reduction, acetylation, glycosylation and demethylation. Applicant's previous studies have shown that B. bassiana can modify several structurally different substrates such as curvularin (macrolactone) and quercetin (flavonoid) to generate new glycosides.

### BRIEF SUMMARY

Applicant has identified a need for new biocatalysts useful for preparing halogenated compounds. The present disclosure in aspects and embodiments addresses this need by providing (i) an Rdc2 halogenase useful for tailoring diverse or complex structures, (ii) methods of substantially isolating or substantially purifying an Rdc2 halogenase, (iii) methods of using an Rdc2 halogenase to provide halogenated products, (iv) halogenated products produced with the use of such a halogenase, and (v) rdc2 gene sequences encoding for an Rdc2 halogenase.

In one embodiment, the present disclosure provides for isolated or purified nucleic acid molecules comprising nucleotide sequences at least 60% 70%, 90%, 95%, 98%, or 100% identical the sequence set forth in SEQ ID NO. 1. In a related embodiment, the present disclosure also provides for nucleic acid molecules that are complimentary to nucleotide sequences at least 60%, 70%, 90%, 95%, 98%, or 100% identical the sequence set forth in SEQ ID NO. 1. Nucleic acid molecules of the present disclosure may comprise an rdc2 gene. Optionally, nucleic acid sequences of the present disclosure may be cDNA, genomic DNA, or synthetic DNA. Additionally, nucleic acid sequences of the present disclosure may be in a single-stranded confirmation. Alternatively, nucleic acid sequences of the present invention may be in a double-stranded configuration. In yet another embodiment, the present disclosure provides for a flavin-dependent Rdc2 halogenase comprising an amino acid sequence at least 60%, 70%, 90%, 95%, 98%, or 100% identical to SEQ ID NO. 2. In embodiments, it is preferable that any of the sequences above provide by an enzyme with Rdc2 halogenating activity. Accordingly, any subunit or altered sequence of the above sequences, whether it is a nucleic acid or amino acid sequence, that ultimately provide for an enzyme with a halogenase activity, are to be considered a functional subunit of the disclosed sequences. In related embodiments, there are provided herein in vitro and in vivo methods for halogenating compounds by using an Rdc2 halogenase of the present invention. Optionally, the in vitro and in vivo methods for halogenation described herein may be part of a sequential biotransformation.

### BRIEF DESCRIPTION OF THE DRAWINGS

FIG. 1 shows expression and purification of the Rdc2 halogenase as set forth in SEQ ID NO. 2.

FIG. 2 shows expression and purification of Fre.

FIG. 3 shows A) HPLC analysis of chlorination of compound 2 catalyzed by an Rdc2 halogenase of SEQ ID NO. 2 (top: reaction mixture; bottom: substrate control); B) HPLC analysis of chlorination of compound 3 with different Fre/Rdc2 ratios; C) Bromination of compound 3 by an Rdc2 of SEQ ID NO 2. All samples were analyzed at 310 nm.

FIG. 4 shows ESI-MS spectrum of compound 2a.

FIG. 5 shows UV spectra of compounds 2 and 2a.

FIG. 6 shows ESI-MS spectrum of compound 3a.

FIG. 7 shows ESI-MS spectrum of compound 3b.

FIG. 8 shows UV spectra of compounds 3, 3a and 3b.

FIG. 9 shows ESI-MS spectrum of compound 3c.

FIG. 10 shows ESI-MS spectrum of compound 3d.

FIG. 11 shows UV spectra of compounds 3, 3c and 3d.

FIG. 12 shows HPLC analysis of chlorination of compound 4 by an Rdc2 of SEQ ID NO. 2. (top: reaction mixture; bottom: substrate control)

FIG. 13 shows ESI-MS spectrum of compound 4a.

FIG. 14 shows UV spectra of compounds 4 and 4a.

FIG. 15 shows HPLC analysis of chlorination of compound 5 by Rdc2 (top: reaction mixture; bottom: substrate control).

FIG. 16 shows ESI-MS spectrum of compound 5a.

FIG. 17 shows UV spectra of compounds 5 and 5a.

FIG. 18 shows HPLC analysis of chlorination of compound 6 by an Rdc2 halogenase (top: reaction mixture; bottom: substrate control).

FIG. 19 shows ESI-MS spectrum of compound 6a.

FIG. 20 shows UV spectra of compounds 6 and 6a.

FIG. 21 illustrates shown selected ¹H—¹H COSY and HMBC correlations for compounds 2a and 3a.

FIG. 22 illustrates sequential halogenations of compound 3 by an Rdc2 halogenase.

FIG. 23 illustrates selected ¹H—¹H COSY and HMBC correlations for compounds 7 and 8.

FIG. 24 illustrates sequential biotransformation of dihydroresorcylide (compound 3) by E. coli/pJZ54 and B. bassiana.

### DETAILED DESCRIPTION

The present disclosure covers enzymes, nucleotide sequences and associated methods useful for halogenating compounds. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

Applicant has identified the strict substrate specificity of flavin-dependent halogenases as an obstacle in the preparation of halogenated compounds. In overcoming this obstacle, applicant has discovered halogenation enzymes, nucleotide sequences and methods useful in producing structurally diverse products of enzymatic halogenation. The present disclosure provides for flavin-dependent Rdc2 halogenases, rdc2 nucleotide sequences encoding for Rdc2 halogenases, methods of using Rdc2 halogenases and compounds produced by the halogenation activity of Rdc2 halogenases. In embodiments, the present disclosure provides for a fungal RAL halogenase from the radicicol biosynthetic gene cluster in P. chlamydosporia. The identified Rdc2 halogenase may be useful as a late tailoring enzyme capable of halogenating various molecules. Optionally, compounds halogenated by a Rdc2 halogenase of the present disclosure may be aromatic compounds, lactones, macrolactones, naturally occurring lactones, or antibiotics. Under sufficient conditions, Rdc2 halogenases of the present disclosure may act as dihalogenases. This unexpected discovery provides for the first time a promising biocatalyst for in vivo or in vitro preparation of both mono- and dihalogenated derivatives from diverse molecules.

In one embodiment, the present disclosure provides for isolated or purified nucleic acid molecules comprising nucleotide sequences at least 60%, 70%, 90%, 95%, 98%, or 100% identical the sequence set forth in SEQ ID NO. 1. In a related embodiment, the present disclosure also provides for nucleic acid molecules that are complimentary to nucleotide sequences at least 60%, 70%, 90%, 95%, 98%, or 100% identical the sequence set forth in SEQ ID NO. 1. Nucleic acid molecules of the present disclosure may comprise an rdc2 gene. Optionally, nucleic acid sequences of the present disclosure may be cDNA, genomic DNA, or synthetic DNA. Additionally, nucleic acid sequences of the present disclosure may be in a single-stranded confirmation. Alternatively, nucleic acid sequences of the present invention may be in a double-stranded configuration. In yet another embodiment, the present disclosure provides for a flavin-dependent Rdc2 halogenase comprising an amino acid sequence at least 60%, 70%, 90%, 95%, 98%, or 100% identical to SEQ ID NO. 2. In related embodiments, there are provided herein in vitro and in vivo methods for halogenating compounds by using an Rdc2 halogenase of the present invention. Optionally, the in vitro and in vivo methods for halogenation described herein may be part of a sequential biotransformation.

Due to the redundancy of the genetic code, and due to the well-known similarities among some amino acids, one skilled in the art would be able to at once envision changes to the sequence of SEQ ID NO. 1 that would not alter the sequence of SEQ ID NO. 2. Additionally, one skilled in the art can envision changes in the sequence of SEQ ID NO. 2 that would not substantially change the functionality of the protein encoded by SEQ ID NO. 2. Furthermore, certain modifications to nucleotide sequences and amino acid sequences, common in the field of biotechnology, could at once be envision by one skilled in the art. Examples of such modifications include, but are not limited to, deletions, additions, fusions, and the introduction or altering of enzyme restriction sites. All of the above modifications and alternative embodiments of the invention are within the scope and spirit of the invention.

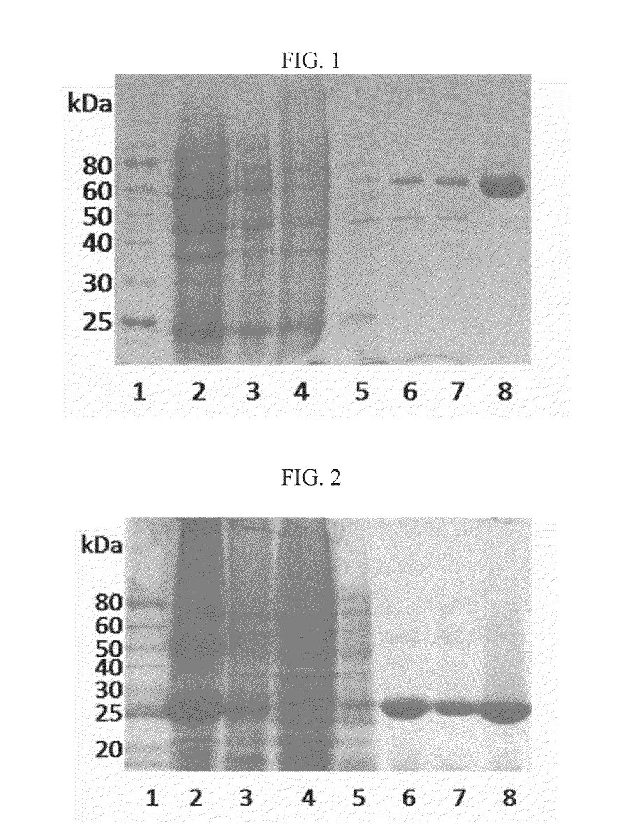

Referring now to FIG. 1, there is shown expression and purification of an isolated Rdc2 halogenase set forth in SEQ ID NO. 2. Shown are (1) protein ladder, (2) soluble fraction, (3) insoluble fraction, (4) flow through, (5) elution fraction by buffer A, (6) elution fraction by buffer A containing 10 mM imidazole, (7) elution fraction by buffer A containing 25 mM imidazole, and (8) elution fraction by buffer A containing 250 mM imidazole.

In some embodiments, rdc2 nucleotide sequences provided for by the present disclosure may be incorporated in various biological vectors. Optionally, the vector may be a plasmid. Vectors containing an rdc2 polynucleotide of the present disclosure may be used to transform competent cells. By way of example, the transformed cell may be a bacterium. In related embodiments, halogenation by in vivo biotransformation may be carried out using cells transformed with an rdc2 nucleic acid molecule of the present disclosure. Transformed cells may be fed substrates for halogenation. Subsequent to the feeding of substrate compounds to the cells, expression of Rdc2 may be induced. The expression of Rdc2 results in the halogenation of the substrate compound. Alternatively, the expression of Rdc2 may result in the halogenation of a derivative or metabolite of the substrate compound.

In one embodiment, without limiting the embodiments of the present disclosure, in vivo halogenation methods of the present disclosure can be carried out by one or more of the following steps. First, inducing a heterologous host transformed with an rdc2 nucleic acid molecule of the present disclosure such that an Rdc2 halogenase is expressed. Heterologous hosts of the present disclosure are intended to include any heterologous host in the field of biotechnology, whether existing now or later developed. By way of example, heterologous hosts of the present invention may include bacteria or yeast. Without limiting the invention, specific examples of heterologous hosts are provided in the various examples of the present disclosure. Second, feeding the induced heterologous host a substrate compound such that the substrate compound may be halogenated by an Rdc2 halogenase of the present disclosure such that the substrate compound. A third step may comprise culturing the heterologous host under sufficient conditions and for a sufficient period of time such that the Rdc2 halogenase carries out halogenation of the substrate compound, to produce a halogenated product. Generally, sufficient conditions and periods of time are any that allow for halogenated product to be produced. Without limiting the invention, specific examples of sufficient conditions and periods of time for in vivo halogenation are provided in the various embodiments and examples of the present disclosure. Varying the conditions and time for halogenation to achieve or optimize halogenation of a specific compound may be desirable, and such variations are within the skill of one in the art. A fourth step may comprise optionally purifying the halogenated product such that the product is 80%, 90%, or 95% pure.

In a related embodiment, the present disclosure provides for heterologous hosts comprising an rdc2 nucleic molecule of the present disclosure and capable of expressing the encoded Rdc2 halogenase. The rdc2 molecule may comprise an expression vector. The expression vector may contain an inducible promoter for inducing expression of the rdc2 transgene. Alternatively, the expression vector may contain a constitutive promoter. Without limiting the invention, the expression vector may be a plasmid. By way of example, the plasmid may be a T7 plasmid.

In another embodiment, in vitro halogenation may be carried out by contacting an Rdc2 halogenase of the present disclosure to a substrate compound in the presence of oxygen, halite, NADH, FAD and a flavin reductase, under sufficient conditions and for a sufficient period of time for the Rdc2 halogenase to halogenate the substrate compound. Without limiting the invention, specific examples of sufficient conditions and periods of time for in vitro halogenation are provided in the various embodiments and examples of the present disclosure. Varying the conditions and time for halogenation to achieve or optimize halogenation of a specific compound may be desirable, and such variations are within the skill of one in the art.

### EXAMPLES

The following examples are illustrative only and are not intended to limit the disclosure in any way. For each example set forth below that utilizes an Rdc2 halogenase of the present disclosure, an Rdc2 halogenase comprising the amino acid sequence as set forth in SEQ. ID NO. 2 was used to carry out halogenation.

### Example 1

### Synthesis of Halogenated Compounds

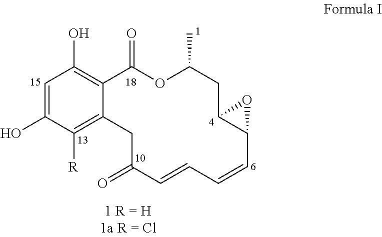

Formula I shows monocillin I (compound 1) and radicicol (compound 1a), which are potent heat shock protein 90 (Hsp90) inhibitors isolated from various fungi. Compound 1a is a chlorine-containing resorcylic acid lactone (RAL). The radicicol biosynthetic gene cluster from two different radicicol producing fungi, Pochonia chlamydosporia and Chaetomium chiversii, have been recently reported, both containing a putative halogenase (rdc2-like or radH-like) that may be involved in the chlorination of the RAL structure, however, neither of these enzymes have been biochemically characterized. Their enzymatic properties remain unknown. Applicant has isolated a new fungal halogenase Rdc2 as set forth in SEQ ID NO. 2 from P. chlamydosporia, useful for structural modification of diverse halogenated products.

Referring now to FIG. 1, to biochemically characterize the Rdc2 halogenase of the present disclosure, the functional enzyme was obtained. It was determined that five introns are present in the rdc2 gene encoding the enzyme. mRNA was extracted from P. chlamydosporia, which was subsequently used for the synthesis of cDNA. The intron-free rdc2 gene was cloned using the cDNA as the template and subsequently ligated into pET28a vector between NdeI and HindIII sites to yield the plasmid pJZ54. The sequencing result of the plasmid confirmed that the cloned rdc2 is intron-free, and revealed that it is unexpectedly longer than the predicted cDNA in GenBank (EU520419). The cloned sequence of the rdc2 gene is shown in SEQ ID NO. 1. The plasmid was then transformed into Escherichia coli BL21-CodonPlus (DE3)-RIL strain (hereafter referred to as RIL) and the protein expression was induced by 200 μM IPTG at 28° C. for 16 h. The N-terminal His-tagged enzyme was purified into homogeneity by Ni-NTA column at a yield of 6.6 mg L⁻¹.

Referring now to FIG. 2, there is shown data demonstrating the successful expression and purification of Fre. BLAST analysis indicated that the Rdc2 of SEQ ID NO. 2 may be a putative flavin-dependent halogenase. Function of flavin-dependent halogenases requires a partner flavin reductase to generate FADH₂ from FAD and NADH. Since such a reductase was absent in the radicicol biosynthetic gene cluster, we chose Fre, a known flavin reductase from E. coli, as the coupling enzyme to test the function of the Rdc2 of SEQ ID NO. 2. Using standard techniques, the fre gene was cloned from the genomic DNA of E. coli BL21 (DE3), inserted into pET28a, and expressed in RIL. The N-terminal His-tagged enzyme was purified by Ni-NTA column at a yield of 10.3 mg L⁻¹.

Still referring to FIG. 2, the following are shown: (1) Protein ladder; (2) Soluble fraction; (3) Insoluble fraction; (4) Flow through; (5) Elution fraction by buffer A; (6) Elution fraction by buffer A containing 10 mM imidazole; (7) Elution fraction by buffer A containing 25 mM imidazole; and (8) Elution fraction by buffer A containing 250 mM imidazole.

With the soluble Rdc2 and Fre enzymes in hand, the catalytic ability of the Rdc2 halogenase to transform compound 1 into compound 1a was determined. Many peaks appeared in the HPLC chromatogram of the reaction mixture, likely due to the instability of the double bonds and epoxide in both compound 1 and compound 1a, in the presence of hypochlorous acid generated during the halogenation reaction. Next, a more stable biosynthetic intermediate or by-product in radicicol biosynthesis was used as a substrate to assay the Rdc2-catalyzed halogenation.

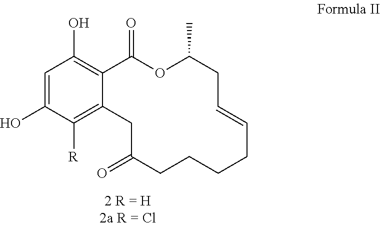

Formula II covers, without limitation, monocillin IV (compound 2) and a halogenated product compound 2a, produced by the Rdc2 halogenase. Monocillin IV was tested as an alternative substrate.

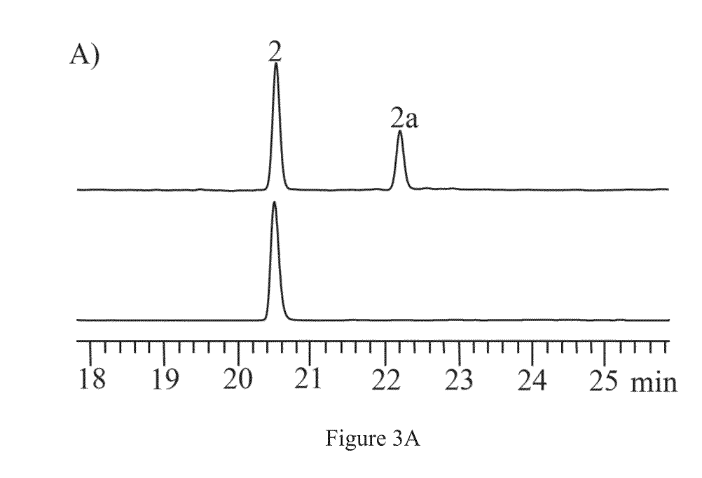

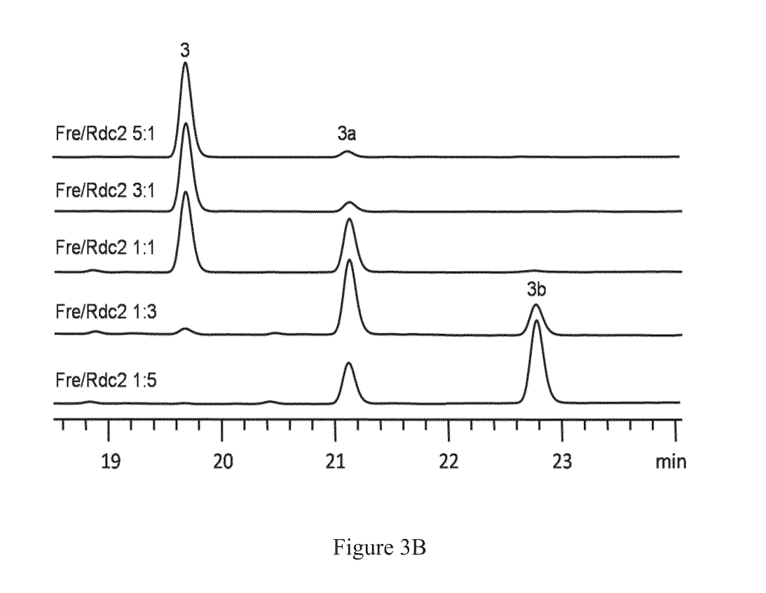

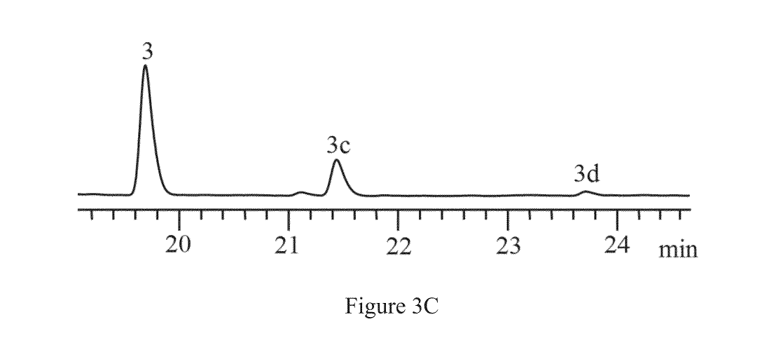

Referring now to FIG. 3a, HPLC analysis of the reaction mixture indicated that the substrate was converted into a compound 2a that has a longer retention time (22.2 min) than the substrate compound 2.

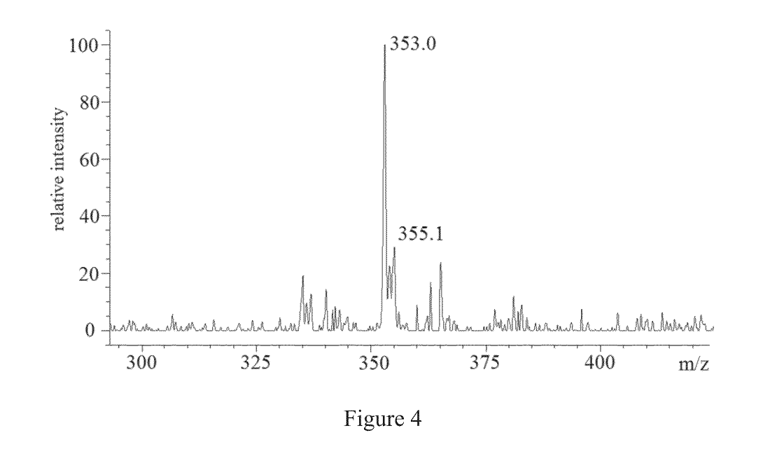

Referring now to FIG. 4, ESI-MS of compound 2a showed the [M+H]⁺ quasimolecular peaks at m/z 353 and 355, respectively, with a ratio of 3:1, which is a characteristic isotope pattern of mono-chlorinated compounds.

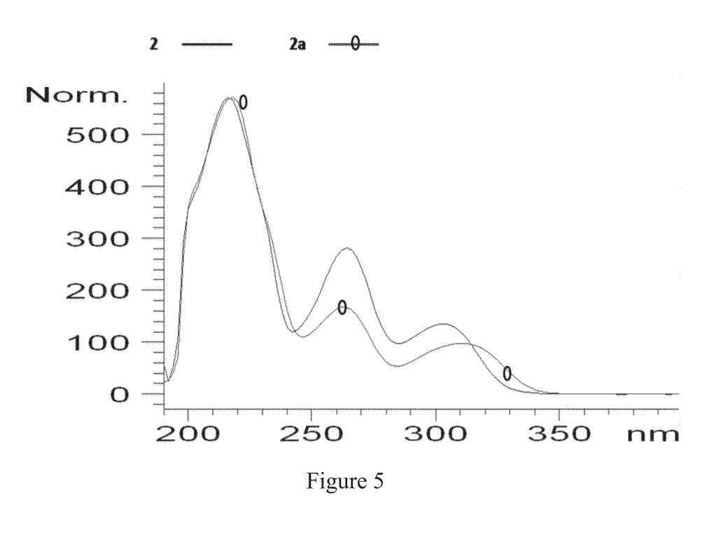

Referring now to FIG. 5, comparing the UV spectrum of compound 2a to that of the substrate compound 2 revealed a bathochromic shift of the UV absorptions, which was likely caused by the chlorination of the substrate compound 2. To obtain sufficient amount of compound 2a for NMR analysis, an in vivo biotransformation method was used to prepare the chlorinated product. 8.2 mg of compound 2 was fed into 1 L of induced E. coli RIL/pJZ54, from which a total of 1.6 mg of compound 2a was purified. ¹H NMR spectrum of compound 2a indicated that only one aromatic proton signal (singlet at δ 6.46, Table 1) is present in compound 2a, suggesting that either 13-H or 15-H was substituted by chlorine. The HMBC correlations of 11-H (δ4.36 and 4.09) to C-13 and C-10 confirmed that compound 2a is 13-chloromonocillin IV. Thus, the Rdc2 halogenase was identified as a flavin-dependent halogenase. The flavin-dependent fungal halogenase was reconstituted in E. coli. RALs are synthesized by cooperative action of two iterative polyketide synthases (PKSs). Results indicated that the Rdc2 halosgenase set forth in SEQ ID NO. 2 may be a dedicated post-PKS tailoring halogenase in radicicol biosynthesis, and may be responsible for the introduction of the chlorine atom after the formation of the RAL structure by the PKSs.

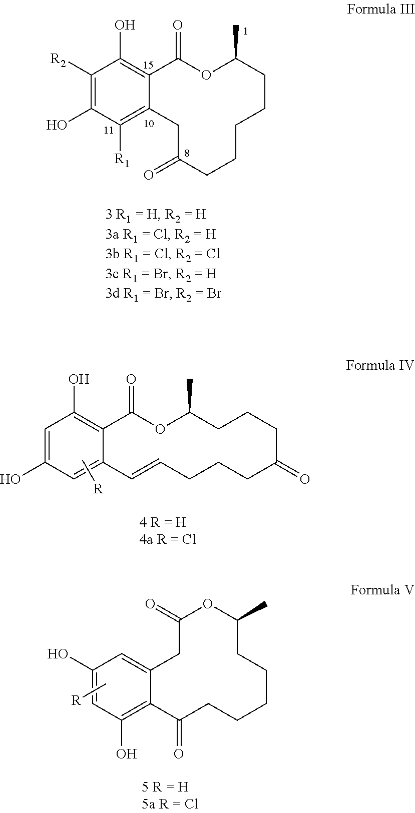

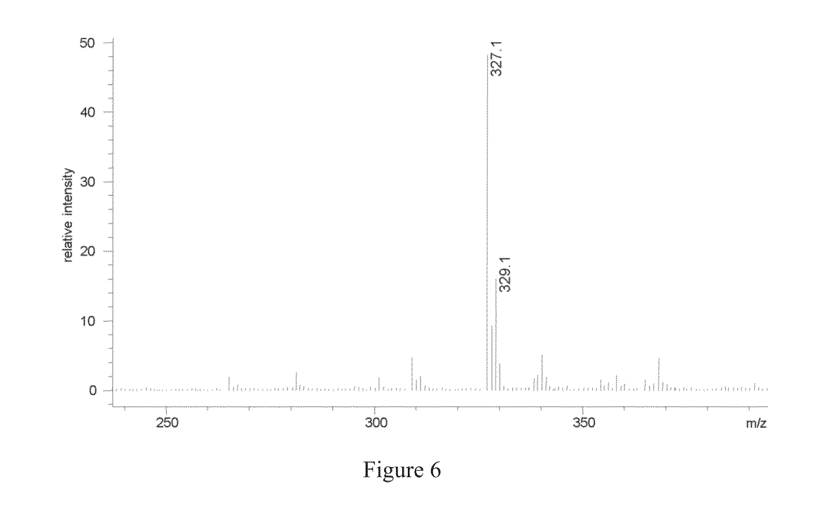

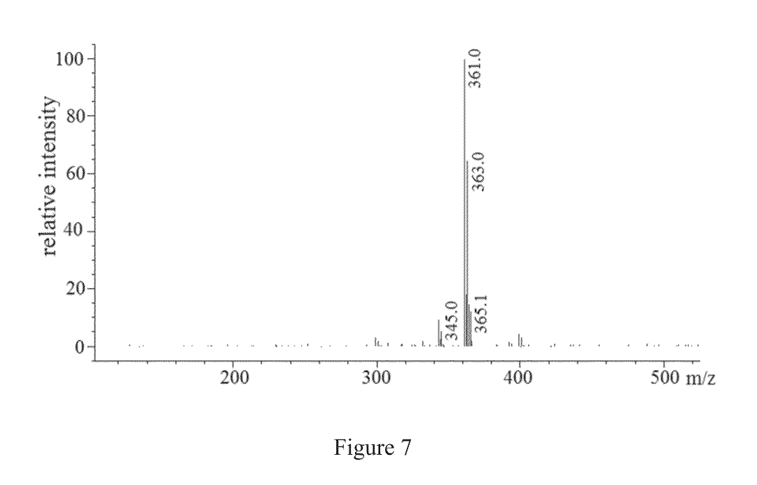

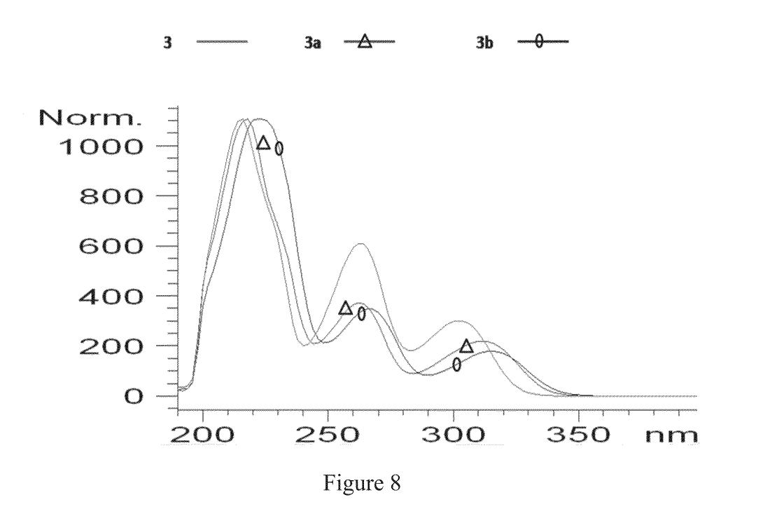

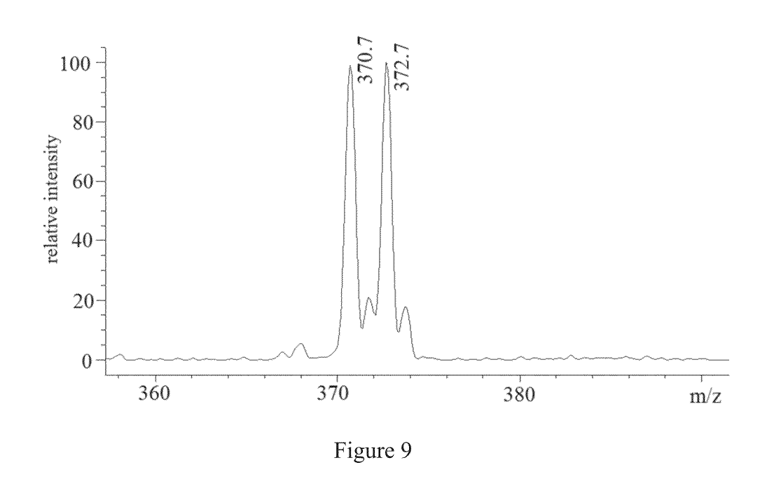

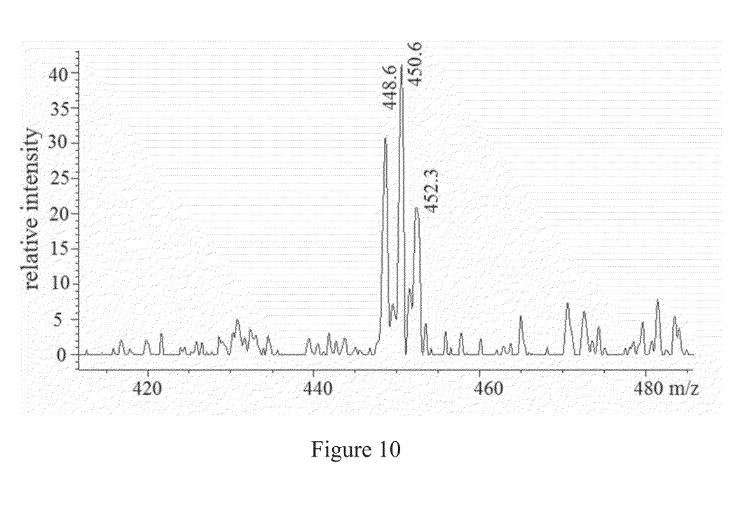

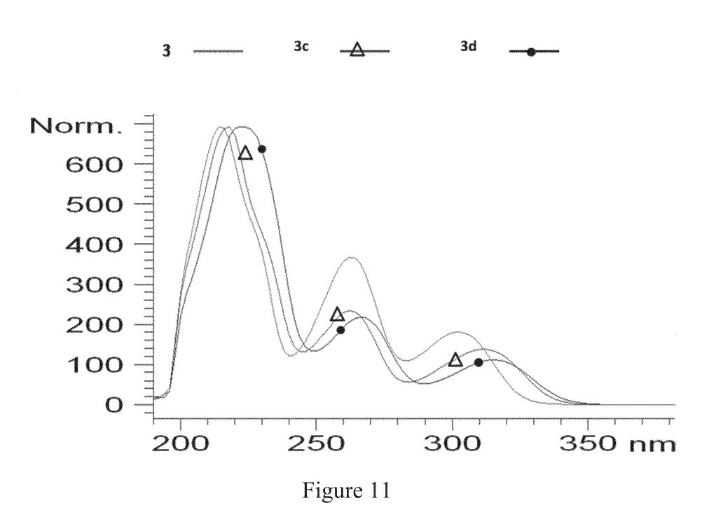

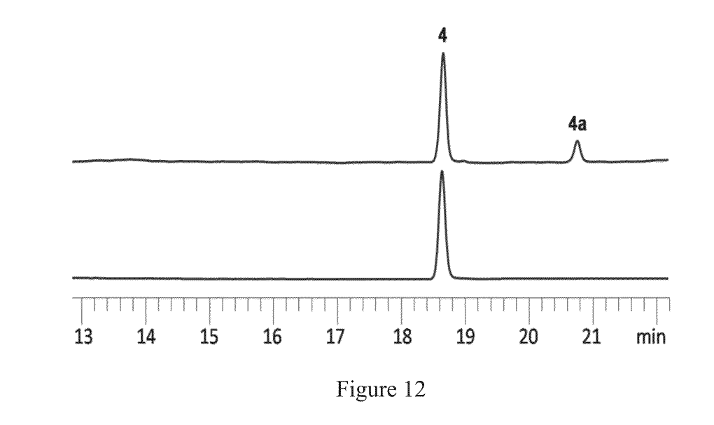

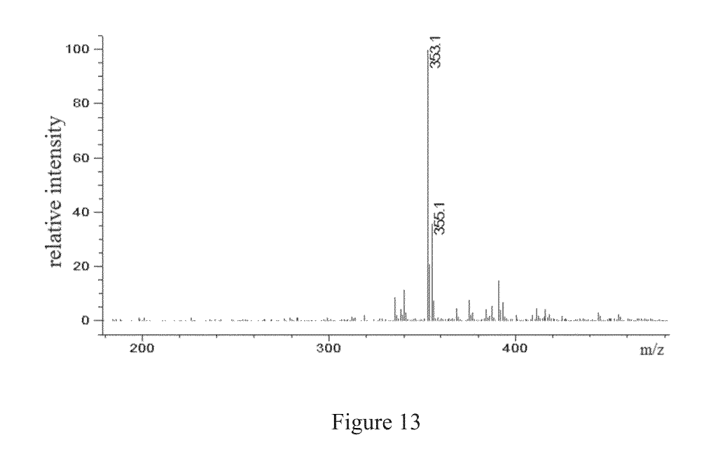

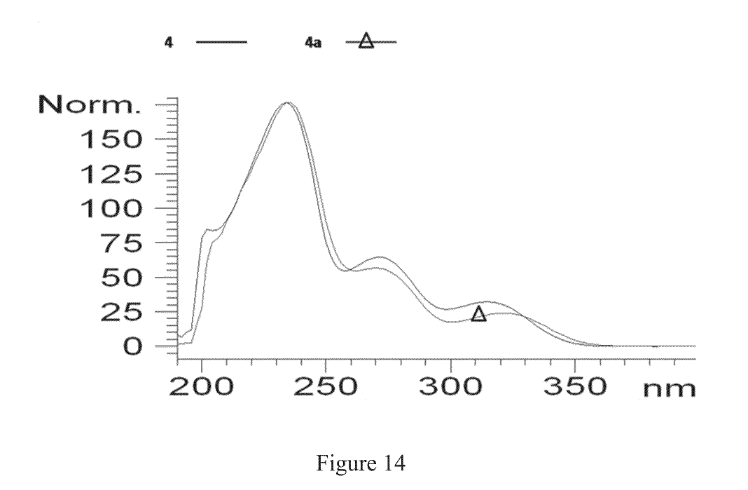

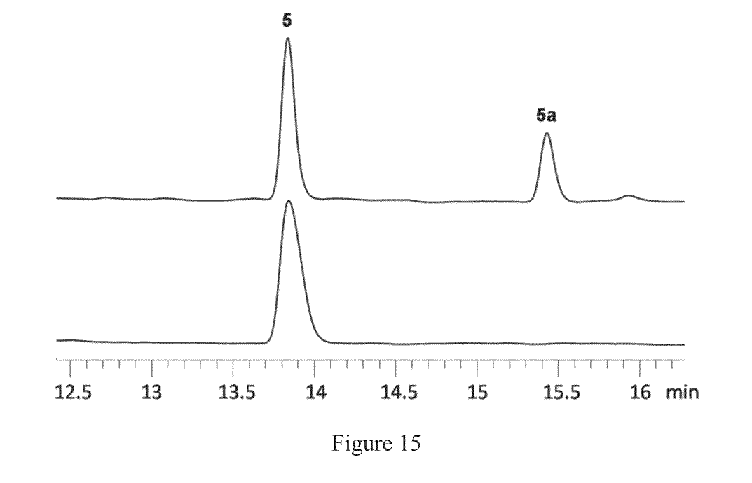

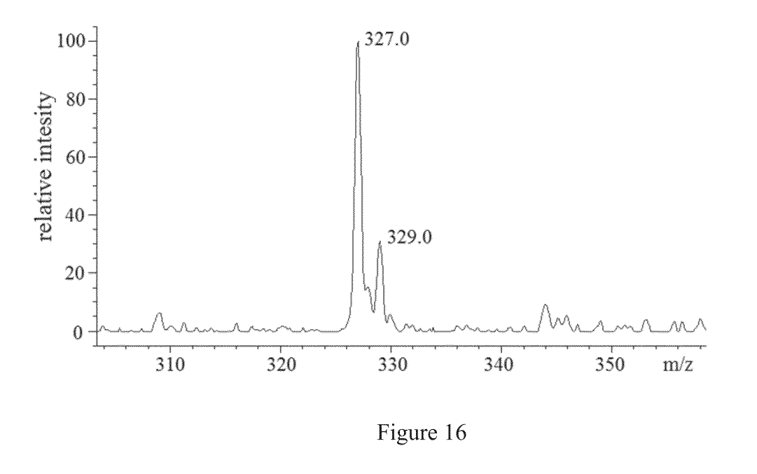

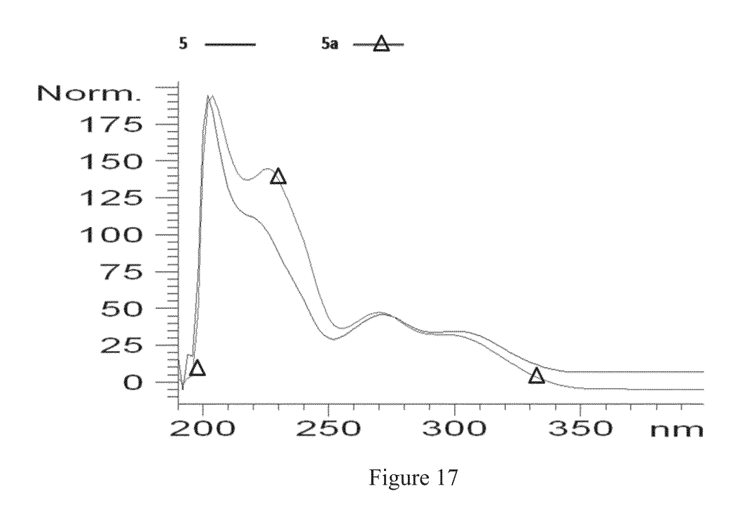

Referring now to FIGS. 6-18, an Rdc2 halogenase of the present disclosure was tested for effectiveness as a halogenating biocatalyst. First, it was examined whether it can function on a variety of natural lactones, including dihydroresorcylide (compound 3), zearalenone (compound 4), and curvularin (compound 6). The results showed that the Rdc2 halogenase can chlorinate all these substrates to the corresponding chlorinated derivative compounds 3a-5a, confirmed by the characteristic [M+H]⁺ ion peaks. It should be noted that both compounds 3 and 5 are a pair of regioisomers with the only structural difference in the position of the lactone and ketone groups. It is thus demonstrated that the Rdc2 halogenase can accept a variety of macrolactones as the substrates to generate chlorinated derivatives.

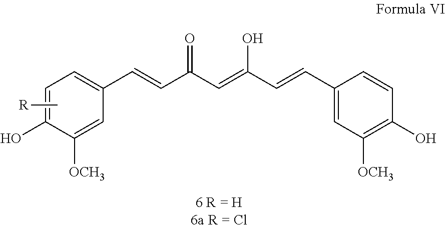

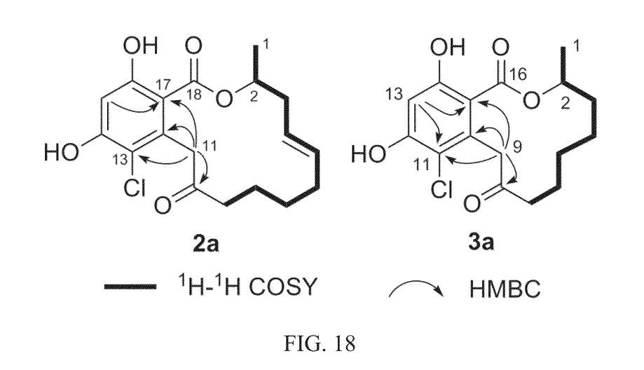

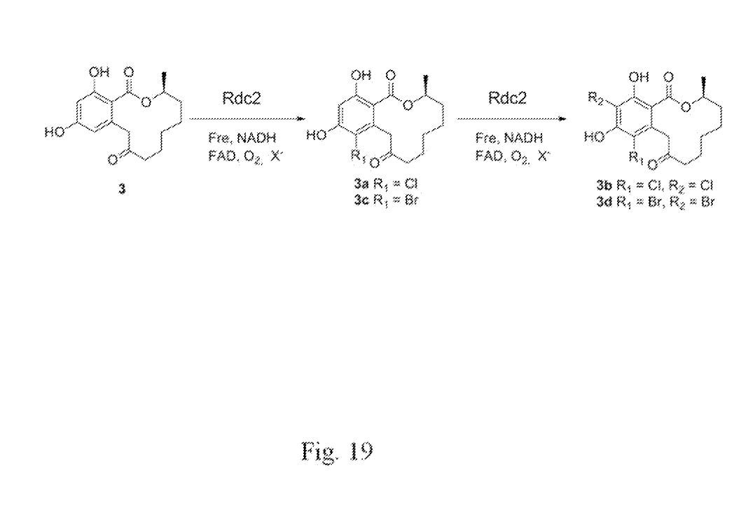

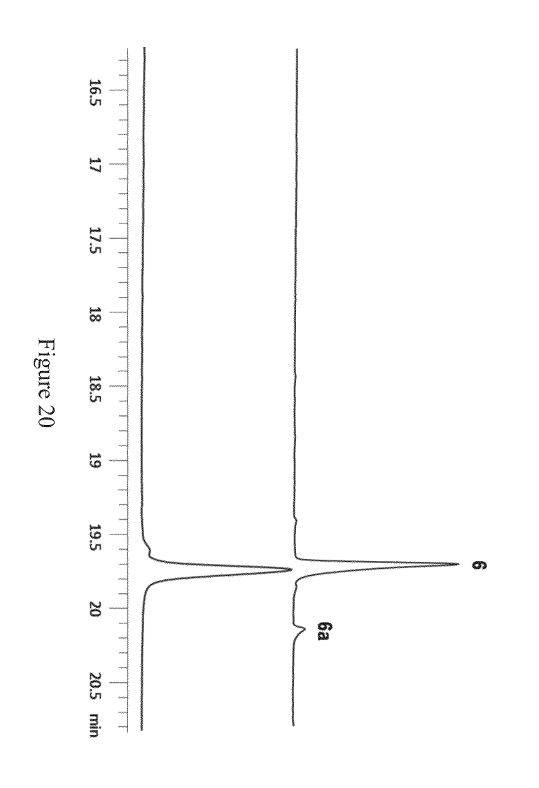

Next, curcumin (compound 6), a well-known bioactive natural product that is synthesized by plant PKSs, was tested as a substrate for halogenation. This compound exhibits many biological properties such as antioxidant and anti-inflammatory activities. Referring now to FIGS. 18-20, LC-MS analysis confirmed that a chlorinated product has been formed at a relatively lower yield compared to the macrolactones. The lower conversion rate might be due to inefficient substrate-enzyme binding. Since compound 6 has a distinct linear structure and totally different biosynthetic origin, chlorination of this compound further confirmed that Rdc2 halogensases of the present disclosure may have broad substrate specificity.

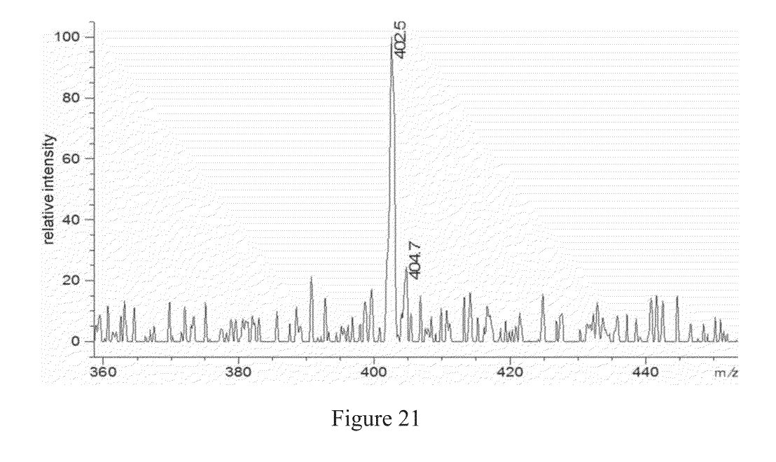

Referring now to FIG. 21, there are shown selected ¹H—¹H COSY and HMBC correlations for compounds 2a and 3a.

TABLE 1
¹H NMR data for compounds 2a, 3a and 3b (in CD₃OD, 300 MHz)

| | ¹H, δ (multiplicities, J) ^{[a]} | | |
|---|---|---|---|
| position | Compound 2a | Compound 3a | Compound 3b |
| 1 | 1.36 (3H, d, 6.5) | 1.31 (3H, d, 6.5) | 1.34 (3H, d, 6.2) |
| 2 | 5.31 (1H, m) | 5.09 (1H, m) | 5.16 (1H, m) |
| 3 | 2.50 (1H, m) | 1.70 (2H, m) | 1.72 (2H, m) |
| | 2.30 (1H, m) | | |
| 4 | 5.50 (1H, m) | 1.52 (1H, m) | 1.51 (1H, m) |
| | | 1.97 (1H, m) | 1.97 (1H, m) |
| 5 | 5.48 (1H, m) | 1.49 (2H, m) | 1.49 (2H, m) |
| 6 | 2.08 (2H, m) | 1.30 (2H, m) | 1.30 (2H, m) |
| 7 | 1.62 (2H, m) | 2.64 (1H, m) | 2.66 (1H, m) |
| | | 2.49 (1H, m) | 2.52 (1H, m) |
| 8 | 1.54 (2H, m) | | |
| 9 | 2.55 (2H, m) | 4.64 (1H, d, 18.5) | 4.65 (1H, d, 18.9) |
| | | 4.50 (1H, d, 18.5) | 4.53 (1H, d, 18.9) |
| 11 | 4.36 (1H, d, 17.5) | | |
| | 4.09 (1H, d, 17.5) | | |
| 13 | | 6.40 (s, 1H) | |
| 15 | 6.46 (1H, s) | | |

^{[a]} J values were reported in Hz.

To investigate the catalytic properties of Rdc2 halogenases of the present disclosure, compound 3 was chosen as the representative substrate. First, compound 3a was prepared through in vivo biotransformation as done for compound 2a. A total of 9.8 mg of compound 3a was prepared in pure form. ESI-MS showed two quasimolecular ion peaks [M+H]⁺at m/z 327 and 329 (3:1), indicating that it is a monochlorinated derivative of compound 3. Similar to compound 2a, only one aromatic proton signal (singlet at δ6.40, Table 1) was observed in the ¹H NMR spectrum of compound 3a. Further 1D and 2D NMR analyses (FIG. 21) confirmed that 11-H in the substrate has been substituted by chlorine. Thus, the structure of compound 3a is 11-chlorodihydroresorcylide.

It has been shown that the ratio of the molar concentrations of a halogenase and its partner flavin reductase will influence the catalytic efficiency. A previous research reported that a ratio of 3:1 of RebF(reductase)/RebH(halogenase) is optimal for halogenation. To achieve the optimal catalytic efficiency for Rdc2 halogenase of the present disclosure, a series of reactions with five different Fre/Rdc2 ratios were examined.

Referring now to FIG. 3B, a Fre/Rdc2 ratio of 5:1 can completely chlorinate compound 3 in 2 hours, indicating that reduction of FAD to FADH₂ by Fre is more efficient than the Rdc2-catalyzed halogenation in the system. More surprisingly, the second product compound 3b appeared when relatively high concentrations of the Rdc2 halogenase of SEQ ID NO. 2 were present. ESI-MS of compound 3b showed the quasimolecular ion [M+H]⁺ peaks at m/z 361, 363 and 365, respectively, with an approximate ratio of 10:6:1 (FIG. 6), suggesting that a dichlorinated derivative was synthesized. We then tried to obtain enough amount of compound 3b for NMR analysis. However, no product was detected in the biotransformation broth of induced E. coli RIL/pJZ54 when compound 3a was fed as the substrate, likely due to the relatively low concentration of the Rdc2 halogenase of SEQ ID NO. 2 in the cells. In an alternative embodiment of the methods disclosed herein, a scaled up in vitro reaction was used to prepare 0.6 mg of 3b from 3a. ¹H NMR analysis (Table 1) revealed the absence of any aromatic protons, confirming that 13-H of compound 3a has also been substituted by chlorine.

Next, compound 2 was tested as the substrate using the same in vitro reaction system for compound 3. Similarly, dichlorinated monocillin IV was detected by LC-MS analysis. The discovery of these dichlorinated products was unexpected, because dichlorinated monocillin I has never been found in nature. Although the role of Rdc2 halogenases of the present disclosure was originally believed to be that of a monohalogenase, corresponding to the mono-chlorination of compound 1a, synthesis of compound 3b has revealed Rdc2 halogenases of the present disclosure may catalyze dichlorination. Accordingly, the present disclosure may provide for an Rdc2 halogenase that can dichlorinate free natural molecules. Rdc2 halogenases of the present disclosure may offer unique modifying enzymes for the preparation of various chlorinated structures. This result also indicated that whereas the functions of biosynthetic enzymes are often assigned based on the structure of intermediates and final products, some enzymes may have additional catalytic properties that could not be interpreted from the original metabolites. The dichlorinated products in the original host may be attributed to the low physiological concentration of the Rdc2 halogenase or possibly strong toxicity of dihalogenated metabolites.

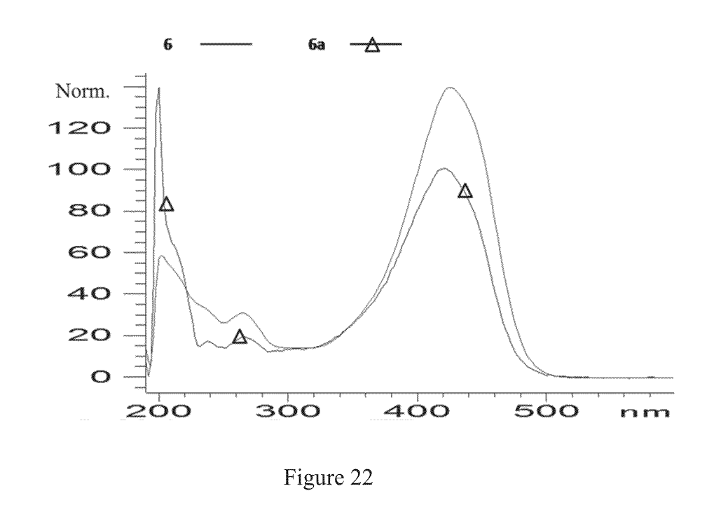

Referring now to FIG. 22, it has been observed that compound 3 was first halogenated at C-11, followed by C-13 halogenation. To understand the reaction sequence, the kinetic parameters of the two chlorination steps of compound 3 were examined. To test C-11 chlorination, compound 3 was used as the substrate and the corresponding k_{cat}/Kₘ ratio is 2.93 min⁻¹ mM⁻¹ (Table 2). Similarly, the k_{cat}/Kₘ value of C-13 chlorination was determined as 0.11 min⁻¹ mM⁻¹ (Table 2) with compound 3a as the substrate. The C-11 halogenation step may be much more efficient and thus favored by Rdc2 halogenases of the present disclosure.

To further explore the potential of the Rdc2 halogenases of the present disclosure, the ability of the Rdc2 halogenase to accept other halogen donors such as bromide and iodide was examined. The results showed that the Rdc2 halogenase can incorporate bromine into compound 3 to yield the corresponding mono-(compound 3c) and dibrominated (compound 3d) products (FIG. 3C and FIG. 22) in a pattern similar to the chlorinations. No iodinated products were observed, which was not surprising considering the large size of iodine atom.

TABLE 2
Steady state kinetic parameters of Rdc2 ^{[a]}

| | C-11 | C-13 |
|---|---|---|
| kinetic parameters | halogenation | halogenation |
| Kₘ (μM) | 281 ± 20 | 846 ± 26 |
| k_{cat} (min⁻¹) | 0.824 ± 0.040 | 0.096 ± 0.004 |
| k_{cat}/Kₘ (min⁻¹ mM⁻¹) | 2.93 | 0.11 |

^{[a]} Data were from three independent experiments.

### Example 2

### Analysis of Products

Products were analyzed and isolated on an Agilent 1200 high performance liquid chromatography (HPLC) instrument. Mass spectra of the compounds were collected by the same HPLC coupled with an Agilent 6130 Single Quad mass spectrometer. Nuclear magnetic resonance (NMR) spectra were recorded in CD3OD on a JEOL instrument (at 300 MHz for ¹H NMR and 75 MHz for ¹³C NMR). The chemical shift (δ) values were referenced to the solvent signals for CD3OD (δ_{H}=3.31) and (δ_{C}=49.15) and were given in parts per million (ppm). The coupling constants (J values) were reported in Hertz (Hz).

E. coli XL 1-Blue and RIL were purchased from Stratagene (La Jolla, Calif., USA) for routine cloning and protein expression, respectively. T4 DNA ligase, 1 kb Plus DNA ladder, SuperScript® III First-Strand cDNA Kit and Platinum Pfx DNA polymerase were from Invitrogen (Carlsbad, Calif., USA). Restriction enzymes and protein ladder were purchased from New England Biolabs (Ipswich, Mass., USA), and pET28a vector was from Novagen (Madison, Wis., USA). P. chlamydosporia ATCC 16683 was obtained from American Type Culture Collection (ATCC). Acremonium zeae NRRL 45893 and Penicillium baradicum NRRL 3754 were obtained from Agricultural Research Service (ARS) of the United State Department of Agriculture. Flavin adenine dinucleotide disodium salt hydrate (FAD), β-nicotinamide adenine dinucleotide, reduced dipotassium salt (NADH), zearalenone (4), and curcumin (6) were purchased from Sigma-Aldrich (St. Louis, Mo., USA). Compound 6 was further purified on HPLC to remove demethoxycurcumin and bisdemethoxycurcumin before it was used as a substrate. Monocillin I (1), radicicol (1a) and monocillin IV (2) were isolated from the potato dextrose agar (PDA) culture of P. chlamydosporia ATCC 16683. Dihydroresorcylide (3) and curvularin (compound 5) were isolated from A. zeae NRRL 45893 and P. baradicum NRRL 3754, respectively.

### Example 3

### Expression and Purification of the Rdc2 as Set Forth in SEQ ID NO. 2

The radicicol producer strain P. chlamydosporia ATCC 16683 was grown in 50 mL of potato dextrose broth (PDB) at 28° C. for 4 days. The culture was filtered and the mycelium was ground in liquid nitrogen. The RNA was extracted from the ground mycelium using a RNeasy Plant Mini Kit from Qiagen. The resulting RNA was used as the template to synthesize the cDNA through a SuperScript® III First-Strand cDNA Kit from Invitrogen. The cDNA was subsequently used as the PCR template to clone the intron-free rdc2 gene. Primers included rdc2-5′-NdeI (AACATATGTCGGTACCCAAGTCTTG) and rdc2-3′-HindIII (AAAAGCTTTCAAACTTTGTTGAGGCCAA). The introduced restriction sites are shown in italics. The PCR product was digested with NdeI and HindIII and inserted into pET28a to yield pJZ54. The plasmid was transformed into E. coli RIL strain for protein expression. For 500 mL of liquid culture, the cells were grown at 37° C. in Luria-Bertani (LB) medium supplemented with 35 μg mL-1 kanamycin and 25 μg mL-1 chloramphenicol to an OD600 of 0.4˜0.6, and then induced by 200 μM isopropyl-1-thio-β-D-galactoside (IPTG) for 16 hours at 28° C. The cells were harvested by centrifugation at 3,500 rpm for 10 minutes, resuspended in 30 mL of cold lysis buffer [20 mM Tris-HCl (pH 7.9), 0.5 M NaCl, 10 mM imidazole, 4° C.] and lysed using sonication on ice. Cellular debris was removed by centrifugation at 20,000 rpm for 45 min at 4° C. Ni-NTA agarose resin (Qiagen) was added to the supernatant (4 mL/L of culture) and the mixture was shaken at 4° C. for 3 hours to ensure the His6-tagged protein was well absorbed. The protein resin mixture was loaded into a gravity flow column and proteins were purified with increasing concentration of imidazole in Buffer A (50 mM Tris-HCl, pH=7.9, 2 mM EDTA, 2 mM DTT). Purified Rdc2 as set forth in SEQ ID NO. 2 was concentrated and exchanged into Buffer A with Centriprep filter devices (Amicon Inc.). The concentration of purified protein was determined by a Commassie Protein Assay Kit from Fisher Scientific. The purity of Rdc2 (58 kDa) was checked on SDS-PAGE (FIG. 1).

### Example 4

### Expression and Purification of Fre

The fre gene was directly cloned form the genomic DNA of E. coli BL21(DE3) using a pair of primers including fre-5′-NdeI (AACATATGACAACCTTAAGCTGTAA) and fre-3′-EcoRI (AAGAATTCTCAGATAAATGCAAACGCAT). The introduced restriction sites are shown in italics. The gene was inserted into pET28a between NdeI and EcoRI sites to yield plasmid pJZ62. The gene was confirmed by sequencing. The expression plasmid pJZ62 was transformed into E. coli RIL strain for protein expression. The expression and purification procedure is same as that for Rdc2 except that the expression temperature is 16° C. The purity of Fre (26 kDa) was checked on SDS-PAGE (FIG. 2).

### Example 5

### Halogenation Assays

A typical halogenation assay mixture (100 μL) consisted of 100 μM FAD, 10 mM NADH, 10 mM NaCl, 0.1 mM substrate, 16 μM Fre, and 16 μM Rdc2 in 100 mM phosphate buffer (pH 7.0). The reaction mixtures were incubated at 28° C. for 2 hours and then quenched with 200 μL of methanol. Substrate controls included all components except the Rdc2 halogenase. The mixtures were briefly vortexed and then centrifuged at 15,000 rpm for 5 min to remove the precipitated proteins before the samples were injected into LC-MS for analysis. The samples were analyzed on an Agilent Single Quad LC-MS using a Zorbax SB-C18 reversed-phase analytical column (5 μm, 150 mm×4.6 mm) at 310 nm, with a flow rate of 1 mL min-1. A gradient of acetonitrile-water system (10-90%) containing 0.1% trifluoroacetic acid (TFA) was programmed over 30 min.

To test other halogen donors, the purified Rdc2 halogenase and Fre were exchanged into phosphate buffer after purification to maximally remove Cl⁻. NaCl in the reaction mixtures was also replaced by NaBr or NaI as the halogen donor. Small amount of mono-chlorinated product 3a may still be observed in these reactions such as the bromination reaction shown in FIG. 3C due to the residual Cl⁻ in the system.

### Example 6

### In Vivo Biosynthesis of 13-chloromonocillin IV (2a) and 11-chlorodihydroresorcylide (3a) in E. coli

E. coli RIL/pJZ54 was grown in 1 L of LB medium and induced as described above. Three hours after IPTG induction, 8.2 mg of 2 (820 μL of a 10 mg mL-1 solution in MeOH) was added and the culture was maintained at 28° C. for an additional 36 hours. The biotransformation broth was then centrifuged to harvest the supernatant and pellet. The resulting pellet and supernatant were extracted for three times with the same volume of MeOH and EtOAc, respectively. The extracts were combined and evaporated under reduced pressure. The residue was dissolved in MeOH and purified by HPLC using the same conditions described above. A total of 1.6 mg of compound 2a was isolated in pure form. ¹³C NMR (CD3OD, 75 MHz): δ 209.9 (C-10), 171.2 (C-18), 162.7 (C-16), 159.3 (C-14), 137.2 (C-12), 135.8 (C-5), 126.6 (C-4), 115.6 (C-13), 108.9 (C-17), 104.0 (C-15), 74.3 (C-2), 47.3 (C-11), 42.2 (C-9), 39.1 (C-3), 33.4 (C-6), 27.0 (C-7), 23.5 (C-8), 19.5 (C-1). ¹H NMR data are listed in Table 1. The signals were assigned based on the 1D and 2D NMR spectra.

The same procedure was used for the preparation of compound 3a. A total of 34 mg of compound 3 was fed into 4 L of induced culture of E. coli RIL/pJZ54, from which 9.8 mg of pure product was isolated by HPLC. ¹³C NMR (CD3OD, 75 MHz): δ 210.8 (C-8), 171.9 (C-16), 163.7 (C-14), 160.7 (C-12), 136.9 (C-10), 117.1 (C-11), 108.2 (C-15), 104.1 (CH-13), 75.5 (CH-2), 47.5 (CH2-9), 43.2 (CH2-7), 33.0 (CH2-3), 28.6 (CH2-5), 22.8 (CH2-6), 22.5 (CH2-4), 19.4 (CH3-1); 1H NMR data are listed in Table 1. The signals were assigned based on the ID and 2D NMR spectra.

### Example 7

### In Vitro Enzymatic Preparation of 11,13-Dichloro-dihydroresorcylide (Compound 3b)

Although in vivo biosynthesis of compound 3b from compound 3a in E. coli was attempted, no product was detected. As an alternative approach to producing compound 3b, the in vitro reaction was scaled up. A 40-mL reaction was set up that contained 100 M FAD, 10 mM NADH, 10 mM NaCl, 0.1 mM substrate, 16 μM Fre, and 16 μM of teh Rdc2 of SEQ ID NO. 2 in 100 mM phosphate buffer (pH 7.0). The reaction mixture was incubated at 28° C. for 16 hours, before it was quenched by 80 mL of MeOH. After brief vortexing, the mixture was centrifuged at 15,000 rpm for 10 minutes. The supernatant was evaporated and re-dissolved in methanol. The product was then purified on the Agilent 1200 HPLC using the same conditions as described for the analysis of small scale reactions. A total of 0.6 mg of compound 3b was isolated in pure form. 1H NMR data of compound 3b are listed in Table 1. The proton signals were assigned by a comparison with compound 3a and the 1H-1H COSY analysis.

### Example 8

### Kinetic Analysis

Kinetic analysis of two chlorination steps of dihydroresorcylide (compound 3) by an Rdc2 halogenase of the present disclosure was carried out. Dihydroresorcylide (compound 3) was chosen as the substrate to study the kinetics of the Rdc2 halogenase. To measure the kₐ, and Kₘ for the C-11 chlorination, a series of 100-μL reaction systems were set up containing 100 μM FAD, 10 mM NADH, 10 mM NaCl, 16 μM Fre, and 16 μM Rdc2 halogenase in 100 mM phosphate buffer (pH 7.0) with varying amount of compound 3 (0.17 to 1.71 mM) in each tube. All the reaction components and Fre were mixed thoroughly and maintained at 28° C. for 2 min. Rdc2 was then added to initiate the reaction. After 30 minutes, the reactions were quenched with 200 μL of MeOH, briefly vortexed, and centrifuged at 15,000 rpm for 5 minutes. Product formation was quantified on the HPLC at 315 nm based on the area of the peaks and standard curve of compound 3a. Reactions were run in triplicate and the steady-state parameters k_{cat} and Kₘ were determined by nonlinear fitting of Michaelis-Menten equation.

For the second halogenations step, C-13 chlorination, the same method was used to determine k_{cat} and Kₘ except that 11-chlorodihydroresorcylide (compound 3a) was used as the substrate (0.03-0.18 mM).

### Example 9

### Sequential Biotransformation

In embodiments, the present disclosure provides for an Rdc2 halogenase that may be used in sequential biotransformation.

Glycosyltransferase from B. bassiana and Rdc2 halogenase may specifically target molecules containing an aromatic ring. The similar substrate preference of these two enzymes and their remarkable broad substrate specificity make it possible to design a sequential biotransformation process for generation of novel halogenated and glycosylated compounds.

To demonstrate the utility of Rdc2 halogenases of the present disclosure in sequential biotransformation, compound 7 was subjected to sequential chlorination and 4′-O-methyl-glucosylation according to the following steps. First, 11-chloro-dihdyroresorcylide (compound 3a) was prepared according to the methods described herein. It has been found that the Rdc2 halogenase, comprising the amino acid sequence set forth in SEQ ID NO. 2, in the original fungus is specifically inhibited by bromide at the transcriptional level, but not influenced in E. coli. Thus, reconstitution and overexpression of this fungal halogenase in a strain of E. coli will not only provide large quantity of the enzyme, but also avoid undesired inhibitory regulation by certain regulatory chemicals or proteins. Briefly, a total of 61.5 mg of compound 3 was fed into induced broth of E. coli BL21-CodonPlus (DE3)-RIL (hereafter referred to as RIL)/pJZ54 for in vivo chlorination and 25.0 mg of compound 3a was purified by preparative HPLC. This compound was subsequently used as the substrate for glycosylation by B. bassiana to yield 5.5 mg of new product compound 8 (14.3% yield).

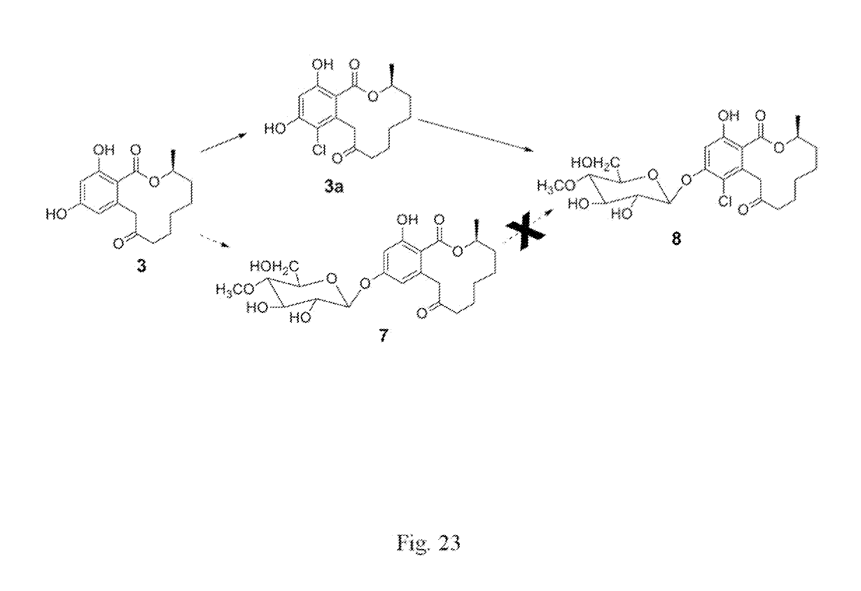

Two [M+Na]⁺ quasimolecular peaks at m/z 525.1 and 527.1 were found in the ratio of 3:1 in the ESIMS of compound 8, showing a characteristic isotope pattern for monochlorinated compounds. Accordingly, the molecular weight of compound 8 can be deduced to be 502, which is 176 mass units more than that of compound 3a, suggesting that a 4′-O-methyl-glucose moiety has been introduced to the substrate. The ¹³C and DEPT NMR spectra revealed that this new product has seven extra oxygenated carbon signals compared to compound 3a, including one CH₃, one CH₂, and five CH signals, indicating that compound 8 is indeed a 4′-O-methyl-glucosylated product, which is further confirmed by the glucose and associated 4′-OCH₃ signals in the ¹H NMR spectrum. These signals were consistent with the previously reported NMR data for 4′-O-methyl-glucose moiety introduced by B. bassiana. To determine what position the 4′-O-methyl-glucose moiety has been attached to, 2D NMR spectra were collected. As shown in FIG. 23, the HMBC spectrum has revealed the correlation of the anomeric proton (1′-H, δ4.94) of the sugar moiety to C-12 (δ 154.4), confirming that the 4′-O-methyl-glucose moiety has been introduced to compound 3a at this position. Therefore, based on the spectral data, compound 8 can be characterized as 1-chloro-4′-O-methyl-12-O-β-D-glucosyl-dihydroresorcylide. Compound 8 is a novel chlorinated and glycosylated derivative of the natural product compound 3. Thus, the present disclosure provides for an Rdc2 halogenase with the ability to participate in biosequential transformation reactions to introduce two functionalities, chlorine atom and 4′-O-methyl-glucose, to the structure of an aromatic compound to construct a new molecule.

In some embodiments, the halogenation step in a sequential biotransformation may be carried out after a first biotransformation step. By way of example, 4′-O-methyl-glucosylation may be followed by chlorination. To demonstrate, compound 7 was fed into the PDB culture of B. bassiana. A new product compound 7 (15.0 mg, 16.4% yield) was synthesized from 57.0 mg of compound 7. ESIMS spectrum of compound 7 contains a quasimolecular ion peak [M+Na]⁺ at m/z 491, indicating that the new product has a molecular weight of 468, which also has a difference of 176 mass units from that of the substrate compound 3. Similarly, the proton and carbon signals for a 4′-O-methyl-glucose moiety were found in the ¹H and ¹³C NMR spectra of compound 7, confirming that compound 7 is formed from 4′-O-methyl-glucosylation of compound 3. Furthermore, the sugar moiety was determined to be linked to 12-OH by the critical HMBC correlation of the anomeric proton (1′-H, δ4.95) to C-12 (δ 163.0) (FIG. 23). Thus, compound 7 was identified as 4′-O-methyl-12-O-β-D-glucosyl-dihydroresorcylide, which is a new derivative of compound 3.

Production of compound 7 demonstrates that B. bassiana can take compound 3 as the substrate to catalyze the expected 4′-O-methyl-glucosylation, further confirming that the dedicated glycosyltransferase was able to accept a wide range of substrates. Both compounds 3 and 3a were glucosylated at the same position (C-12) with comparable yields, indicating that a chlorine atom at C-11 had no obvious effect on the selectivity and efficiency of the glycosyltransferase.

Since both the Rdc2 halogenase and the dedicated glycosyltransferase possess broad substrate specificity; the next question to be answered is whether the sequence of the two biotransformation steps is interchangeable. Because compound 3 was converted into compound 7 through 4′-O-methyl-glucosylation, an experiment was conducted to test whether compound 7 can be chlorinated to afford compound 8. To this end, compound 7 was incubated with the induced broth of E. coli RIL/pJZ54. However, compound 8 was not detected in the biotransformation broth. This revealed that the halogenase cannot take compound 7 as the substrate to perform the 11-chlorination, likely because the introduced sugar moiety interferes with the substrate-enzyme binding. Therefore, it may be important to precisely arrange the reaction sequence in order to use sequential biotransformation for structural modification, which should be based on the substrate specificity of the involved enzymes.

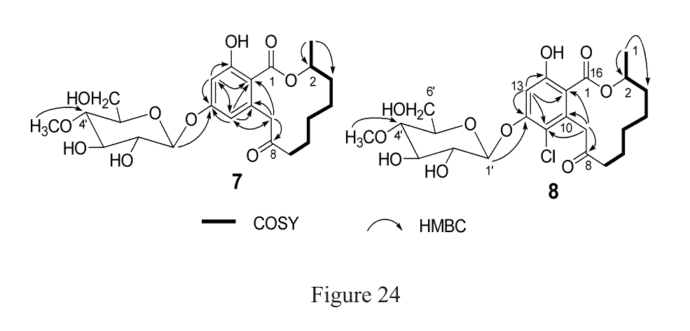

Referring now to FIG. 24, combinatorial or sequential biocatalysis is a useful technology for synthesizing new molecules by taking advantage of particular catalytic properties of enzymes and microbial strains. The present disclosure provides for a sequential biotransformation process consisting of chlorination and 4′-O-methyl-glucosylation. Through the sequential actions of the engineered E. coli RIL/pJZ54 strain and B. bassiana, the present disclosure provides methods to generate a novel chlorinated and glycosylated derivative of a compound, for example, compound 7. Two routes (FIG. 24) have been attempted to synthesize this new product. In the upper route, compound 3 was first to chlorinated at C-11 and then glycosylated at C-12 to give rise to compound 8. However, although the step of 4′-O-methyl-glucosylation of compound 3 in the lower route worked and yielded a new product compound 7, chlorination of this product into compound 8 was not successful. Therefore, the novel chlorinated and glycosylated derivative compound 8 can only be prepared through the upper route.

The present disclosure provides examples of preparation of novel molecules through sequential biotransformation using wide type and engineered microbial strains. By way of example, compound 5 is a regioisomer of compound 3, with the only structural difference in the position of the lactone and ketone groups. This compound can be accepted as the substrate by both Rdc2 halogenases of the present disclosure and the glycosyltransferase. Thus, it is expected that this approach can be used to generate a similar chlorinated and glycosylated derivative of compound 5. Chlorine has been found to be a critical structural component in many bioactive natural products such as vancomycin, and introduction of a chlorine atom into the substrates may generate new biological activities for the derivatives. Additionally, if any of the products produced by chlorination demonstrate biological activity, glycosylation may improve the water solubility of the chlorinated compounds. Without limiting the invention, many macrolactones have been found to possess promising biological activities such as antifungal and anticancer properties. In embodiments, the present disclosure provides for an effective tool to generate novel bioactive molecules from other macrolactones and structurally similar natural products.

To further assist those interested in carrying out sequention biotransformations as described herein, the following methods are provided. These methods are provided as guidance only, and are not necessarily limiting to the invention.

Products from sequential transformation were analyzed and purified on an Agilent 1200 high performance liquid chromatography (HPLC) instrument. NMR, JEOL instrument (300 MHz for ¹H and 75 MHz for ¹³C NMR); MS, Agilent 6130 LC-MS.

B. bassiana ATCC 7159 was purchased from American Type Culture Collection (ATCC). A. aeae NRRL 45893 was obtained from Agricultural Research Service (ARS) of the United State Department of Agriculture. E. coli RIL was from Stratagene (La Jolla, Calif., USA) for heterologous expression of the Rdc2 of SEQ ID NO. 2. Potato dextrose agar (PDA), potato dextrose broth (PDB) and Luria-Bertani (LB) media were purchased from BD Biosciences (Franklin Lakes, N.J., USA). Both B. bassiana and A. aeae were cultured at 30° C., while E. coli was routinely grown in LB medium at 37° C.

Substrate was isolated from A. aeae NRRL 45893. A. aeae NRRL 45893 was grown in 30 Petri dishes, each containing 30 mL of PDA medium. After 7 days, the culture was extracted with 1 L of methanol three times. The methanol extract was concentrated under reduced pressure and separated on an Agilent 1200 HPLC using an Agilent reverse-phase Zorbax SB-C18 preparative column (21.2×150 mm, 5 μm). A gradient system of methanol-water was programmed from 80-90% (v/v) from 0 to 10 min and 90-95% (v/v) from 10 to 20 min at a flow rate of 3 mL/min. The compound was detected at 310 nm and collected at 16.1 min. A total of 124.8 mg of compound 7 was purified from the extract and subsequently used as the substrate for biotransformation.

Compound 8 was produced through sequential biotransformation. Construction of pJZ54 by ligation of the rdc2 comprising the polynucleotide set forth in SEQ ID NO. 1 into pET28a vector is as described above. The plasmid was transformed into E. coli RIL to express the halogenase. E. coli RIL/pJZ54 was grown in 8 L of LB medium supplemented with 35 μg/mL kanamycin and 25 μg/mL chloramphenicol at 37° C. to an OD₆₀₀ of 0.5, which was then induced by 200 μM IPTG at 28° C. Three hours after induction, 61.5 mg of compound 3 was added and the broth was maintained at 28° C. and 250 rpm for an additional 36 h. After centrifugation, the supernatant was extracted with 6 L of ethyl acetate three times and the cells with 1 L of methanol. Both extracts were combined and evaporated under reduced pressure. A total of 25.0 mg of compound 3a was purified from the extract on HPLC using the same preparative conditions as those for compound 3. The retention time of compound 3a is 17.5 min.

B. bassiana ATCC 7159 was grown in 2 L of PDB medium at 250 rpm and 30° C. for 3 days. Compound 3a was then added into the culture and incubated under the same conditions for an additional 5 days. The biotransformation broth was then harvested by filtration. The filtrate was extracted with 2 L of ethyl acetate and mycelium with 500 mL of methanol three times. Both extracts were combined and evaporated under reduced pressure. Compound 8 was purified using an Agilent Zorbax SB-C18 analytical column (4.6×150 mm, 5 μm). A gradient system of acetonitrile-water, each containing 0.1% trifluoroacetic acid (TFA), was programmed from 5-95% (v/v) over 30 min at a flow rate of 1 mL/min. The product was detected at 310 nm and collected at 14.8 min. A total of 5.5 mg of compound 8 was isolated in pure form. Its structure was determined on the basis of the spectral data.

### 11-chloro-4′-O-methyl-12-O-β-D-glucosyl-dihydro-resorcylide (Compound 8) White Powder

¹H NMR (DMSO-d₆, 300 MHz): δ 10.5 (1H, brs. OH-14), 6.69 (1H, s, H-13), 5.08 (1H, m, H-2), 4.94 (1H, d, J=7.9 Hz, H-1′), 4.19 (1H, d, J=18.5 Hz, H-9a), 4.08 (1H, d, J=18.5 Hz, H-9b), 3.74 (1H, m, H-6′a), 3.64 (1H, m, H-6′b), 3.46 (3H, s, OCH₃-4′), 3.46 (1H, m, H-5′), 3.44 (1H, t, J=9.0 Hz, H-3), 3.29 (1H, t, J=8.1 Hz, H-2′), 3.09 (1H, t, J=9.3 Hz, H-4′), 2.55 (1H, m, H-7a), 2.37 (1H, m, H-7b), 1.71 (3H, m, H-3a and H-6), 1.48 (1H, m, H-3b), 1.43 (1H, m, H-4-a), 1.31 (2H, m, H-5), 1.27 (1H, m, H-4-b), 1.17 (3H, d, J=6.3 Hz, CH₃-1).

¹³C NMR (DMSO-d₆, 75 MHz): δ 206.5 (C-8), 167.3 (C-16), 155.9 (C-14), 154.4 (C-12), 132.7 (C-10), 115.2 (C-11), 114.2 (C-15), 102.1 (C-13), 99.7 (C-1′), 78.6 (C-4′), 76.3 (C-3′), 75.7 (C-5′), 73.3 (C-2′), 72.4 (C-2), 59.8 (C-6′), 59.7 (OCH₃-4′), 44.5 (C-9), 41.6 (C-7), 32.0 (C-3), 26.8 (C-5), 22.1 (C-6), 21.7 (C-4), 19.5 (C-1). ESIMS (+): m/z 525.1 and 527.1 (3:1) [M+Na]⁺.

4′-O-Methyl-glucosylation of compound 1 by B. bassiana ATCC 7159 to yield 7: B. bassiana ATCC 7159 was also used to biotransform 57.0 mg of compound 3. The fungal strain was grown in 2 L of PDB medium under the same conditions and processed as described above. A new product compound 7 was purified using the Agilent Zorbax SB-C18 preparative column (21.2×150 mm, 5 μm). A gradient of methanol (A)-water (B) system (both containing 0.1% TFA) was programmed as below to purify compound 7. The flow rate was 5 mL/min and the peaks were monitored at 310 nm. Compound 7 was collected at 10.2 min. A total of 15.0 mg of compound 7 was obtained in pure form. The structure of compound 7 was determined on the basis of the spectral data.

TABLE 3
HPLC solvent system gradient.

| Time (min) | A % (0.1% TFA) |
|---|---|
| 0 | 70 |
| 15 | 90 |
| 16 | 100 |
| 30 | 100 |

### 4′-O-methyl-12-O-β-D-glucosyl-dihydroresorcylide (Compound 7) White Powder

¹H NMR (CD₃OD, 300 MHz): δ 6.56 (1H, d, J=2.4 Hz, H-13), 6.38 (1H, d, J=2.4 Hz, H-11), 5.16 (1H, m, H-2), 4.95 (1H, d, J=7.6 Hz, H-1′), 4.72 (1H, d, J=18.5 Hz, H-9a), 3.89 (1H, d, J=18.5 Hz, H-9b), 3.85 (1H, dd, J=12.0, 1.7 Hz, H-6′a), 3.70 (1H, m. H-6′b), 3.59 (3H, s OCH₃-4′), 3.57 (1H, t, J=9.0 Hz, H-3), 3.44 (1H, m, H-2′ and H-5′), 3.19 (1H, t, J=9.3 Hz, H-4′), 2.69 (1H, m, H-7a), 2.35 (1H, m, H-7b), 2.01 (1H, m, H-6a), 1.82 (1H, m, H-6b), 1.66 (2H, m, H-3), 1.51 (2H, m, H-4), 1.49 (2H, m, H-5), 1.31 (3H, d, J=6.5 Hz, CH₃-1).

### Example 10

### Calculating Percent Identity

In various embodiments and examples of the nucleic acid molecules and amino acid sequences described herein, percent identities can be calculated by means standard in the art. A nucleotide blast or amino acid blast may be used in aligning and calculating the percent identities of the sequences. Sequence alignment and percent identities, as used herein, are calculated as follows. General parameters for the nucleotide blast include a max target sequence of 100, an automatic adjustment for short input sequences, an expect threshold of 10, a word size of 28, a match/mismatch score of 1/−2, and linear gap costs. General parameters for a protein blast include a max target sequence of 100, and adjustment for short queries, an expect threshold of 10, a word size of 3, and no max matches in a query range. Scoring parameters include the use of an established matrix. For example, the matrix may be BLOSUM62. Gap costs are Existence: 11 Extension: 1. Compositional adjustments include a conditional compositional score matrix adjustment.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. It will be appreciated that several of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the spirit of the invention. The invention should therefore not be limited by the above described embodiment, method, and examples, but by all embodiments and methods within the scope and spirit of the invention.

I claim:

1. An isolated enzyme comprising an amino acid sequence selected from a group consisting of (i) the amino acid sequence set forth in SEQ ID NO. 2; (ii) an amino acid sequence that has at least 95% identity to the amino acid sequence set forth in SEQ ID NO. 2; or (iii) a functional subunit of the amino acid sequence set forth in SEQ ID NO. 2.

2. An isolated enzyme of claim 1, further comprising an enzyme having halogenase activity.

3. An isolated enzyme of claim 1, further comprising a flavin-dependent halogenase.

4. An isolated enzyme of claim 1, further comprising an enzyme encoded by the nucleic acid molecule sequence set forth in SEQ ID NO: 1.

5. An isolated nucleic acid molecule comprising a sequence that encodes a halogenase enzyme having an amino acid sequence selected from a group consisting of (i) the sequence set forth in SEQ ID NO. 2; or (ii) a sequence with at least 95% identity to SEQ ID NO. 2.

6. An isolated nucleic acid molecule of claim 5, further comprising a nucleic acid molecule that encodes an enzyme having halogenase activity.

7. An isolated nucleic acid molecule of claim 5, further comprising a nucleic acid molecule encoding a flavin-dependent halogenase.

8. An isolated nucleic acid molecule of claim 5, further comprising a nucleic acid sequence selected from a group consisting of nucleic acid sequences (i) at least 90% identical, (ii) at least 95% identical, and (iii) 100% identical to SEQ ID NO: 1.

9. An isolated nucleic acid of claim 5, further comprising a nucleic acid encoding a flavin-dependent halogenase.

10. An isolated nucleic acid molecule of claim 5, further comprising a vector.

11. An isolated nucleic acid molecule of claim 9, wherein the vector is a plasmid.

12. An in vivo process for halogenating a compound, comprising the following steps (1) inducing a heterologous host to express an Rdc2 halogenase, (2) feeding an aromatic substrate compound to the induced heterologous host (iii) allowing the Rdc2 to halogenate the substrate compound, a derivative of the substrate compound, or a metabolite of the substrate compound.

13. The process of claim 12, further comprising, a sequential in vivo biotransformation.

14. The process of claim 12, wherein the substrate is an aromatic compound.

15. The process of claim 12, wherein the substrate is a macrolactone.

16. The process of claim 12, wherein the substrate is a natural lactone.

17. The process of claim 12, wherein the substrate is an antibiotic.

18. An in vitro process for halogenating a compound comprising contacting a substrate compound with an Rdc2 halogenase.

19. The process of claim 18, wherein the substrate is an aromatic compound.

20. The process of claim 18, wherein the substrate is a macrolactone.

21. The process of claim 18, wherein the substrate is a natural lactone.

22. The process of claim 18, wherein the substrate is an antibiotic.


SEQUENCE LISTING (2 sequences)

SEQ ID NO: 1 - DNA, 1602 bp, Pochonia chlamydosporia
atgtcggtacccaagtcttgtacgattttggtggctggtggtggccctgccggctcgtatgctgctgcagccctggctcgagagggcaatgatgttgtgcttctcgaagcggatcaacatcctcgctatcatatcggcgagagtatgctcccgtcgttgagaccgctgctgcgatttatcgatctcgaagataagtttgatgcacatggatttcagaaaaagctcggtgccgctttcaagttgacttccaagcgcgaaggatacaccgatttcatcgcagcccacggccctagaggatactcctggaacgtggtacgctccgaatccgacgaaatcctcttcaaccatgctcgcgaaagcggcgcccaggccttccaaggcatcaagataaacgcaatcgaattcgaaccatacgaggaagagtaccccaacggagaaaaagtggcaaatcccgggaagcccacgtccgcaaaatggtcgtccaaggatggcagttctggggacatcgccttcaagtatttggtcgacgcgacgggccggattggcatcatgtccacaaagtatctcaagaatcggcactacaacgagggtttaaaaaacctggccatctggggctattacaagaacaacataccttgggcgcagggtacacccagagagaatcagcccttcttcgaaggcatgcgagacggcgctggctggtgctggaccattcctttacacaacggcacagtctcagtcggagcggtcatgcgaaaagatctcttcttcgaaaagaagaagagtcttggtgaaaatgccaccaacacccaaataatggccgaatgcatgaaactatgtcccacgataggagaactgctcgcgccagccgaattggtgtcggacatcaagcaagctacagactattcgtacagcgcgaccgcctatgccgggcccaacttccgcatcgtcggagacgcaggctgcttcatcgatccattcttctcttcgggccatcacttggctgttgccggtgcccttgcggccgccgtctccataaacgcatccatcaaaggcgactgcaccgagtacgaagcctccagatggcacgcaaagaaggtagacgagggttacacattgttcctgctcgtggtcatggctgccctcaagcaaatcaggatgcaggagaacccagtgctgagcgacgttgatgaagacggctttgacagggcattccagttcctcagaccagttatccaaggctccgggtcttcagaggcggttaagaagttcacaaaggaggacgtcgcgcaaacgattgactttgctgtgcatgcgctcaataacatggcggaactggacatggatatcccggagcacatgatcaatggggacaaggagggcgagaatggtgtgacgaatggcaacaatggggctgcaaagacggctggacttgcgtcaaacatggagaagctcacgaatgatgaggagaaggttctcaatggtcttcgtattcttggcaaggctgcgcctggaggaactctggcggactttgagggcactgcaatcgatggactgatgccgagattggaacatggcaaacttggcctcaacaaagtttga

SEQ ID NO: 2 - protein, 533 aa, Pochonia chlamydosporia
MSVPKSCTILVAGGGPAGSYAAAALAREGNDVVLLEADQHPRYHIGESMLPSLRPLLRFIDLEDKFDAHGFQKKLGAAFKLTSKREGYTDFIAAHGPRGYSWNVVRSESDEILFNHARESGAQAFQGIKINAIEFEPYEEEYPNGEKVANPGKPTSAKWSSKDGSSGDIAFKYLVDATGRIGIMSTKYLKNRHYNEGLKNLAIWGYYKNNIPWAQGTPRENQPFFEGMRDGAGWCWTIPLHNGTVSVGAVMRKDLFFEKKKSLGENATNTQIMAECMKLCPTIGELLAPAELVSDIKQATDYSYSATAYAGPNFRIVGDAGCFIDPFFSSGHHLAVAGALAAAVSINASIKGDCTEYEASRWHAKKVDEGYTLFLLVVMAALKQIRMQENPVLSDVDEDGFDRAFQFLRPVIQGSGSSEAVKKFTKEDVAQTIDFAVHALNNMAELDMDIPEHMINGDKEGENGVTNGNNGAAKTAGLASNMEKLTNDEEKVLNGLRILGKAAPGGTLADFEGTAIDGLMPRLEHGKLGLNKV